(19) United States
(12) Patent Application Publication
(10) Pub. No.: US 2019/0143127 A1
(43) Pub. Date: May 16, 2019

(54) HOUSING COMPONENT AND METHOD FOR MANUFACTURING SAME

(71) Applicant: FRAUNHOFER-GESELLSCHAFT ZUR FÔRDERUNG DER ANGEWANDTEN FORSCHUNG E.V., München (DE)

(72) Inventors: Sebastian WINKLER, Oberlungwitz (DE); Jan EDELMANN, Chemnitz (DE); Patrick MANTHEI, Chemnitz (DE)

(21) Appl. No.: 16/300,352

(22) PCT Filed: May 11, 2017
(86) PCT No.: PCT/EP2017/061358

§ 371 Date: Nov. 9, 2018

(30) Foreign Application Priority Data
May 11, 2016 (DE) 10 2016 208 065.2

(51) Int. Cl. A61N 1/372; A61N 1/375; A61L 27/06; A61L 27/10

(52) U.S. Cl. CPC A61N 1/37223; A61N 1/37512; A61B 5/076; A61L 27/10; A61L 27/06

(57) ABSTRACT

Housing component, in particular for implantable devices, comprising at least one housing wall section that includes a window-type opening for conducting electromagnetic waves therethrough, said window-type opening being hermetically sealed as a result of a blank being shaped that is made of material permeable to electromagnetic waves.

The present invention relates to a housing component, in particular for implantable devices, as well as to a method for manufacturing same. The invention further relates to a housing as well as an implantable device comprising said housing component.

It is known to use implantable devices for medical applications, in particular for diagnostic and therapeutic measures for the treatment of various human diseases. These include, for example, heart diseases or neurological diseases. One of the longest known and most significant examples of implantable devices is the pacemaker. In addition, implantable devices that are not directly used to treat diseases, such as the identification of animals using implanted microchips, are gaining in importance.

Implantable devices of this kind are usually equipped with active electronic components. In many cases it is thereby necessary that the active electronic components—so-called intracorporeal components—arranged inside the implantable device and thus inside the body communicate with electronic components outside the body—so-called extracorporeal components. At the same time, there is a need to hermetically encapsulate the active electronic components of the implantable device, thus, the intracorporeal components, due to their toxicity, in order to prevent poisoning of the respective body.

Communication between the intracorporeal and extracorporeal electronic components may, for example, be wired, however, this is disadvantageous in terms of hermetic encapsulation. In addition, it is possible to implement communication by transmitting electromagnetic waves. However, wireless data transmission between intracorporeal and extracorporeal electronics requires permeability to electromagnetic waves of all media and components between the respective transmitter and receiver.

It is generally possible to use glass as the sole encapsulation material so that on the one hand, a complete encapsulation of the intracorporeal electronic components and on the other hand, a sufficient permeability of the encapsulation material for communication between transmitter and receiver is ensured. This type of encapsulation is used, for example, in identification chips for animals. In addition to their use in the human body, such systems are also known in the field of cashless payment. However, such implanted chips enclosed in glass are relatively small. Due to their small size and largely symmetrical outer shape, for example, due to their spherical configuration, the risk of breakage for such glass encapsulations is relatively low. With the size of the implant, the risk of breakage increases so that, for example, for larger implants, such as pacemakers, glass cannot be considered as an encapsulation material.

For hermetic encapsulations with higher stability requirements, metallic materials, in particular titanium or titanium alloys, are usually used. Thus, the use of titanium for the encapsulation of electronic components is associated in particular with the advantage of high biocompatibility. Titanium is not recognized by the human body as a foreign material and, therefore, does not trigger any undesirable reactions. At the same time, titanium ensures a high degree of mechanical stability. On the other hand, titanium is impermeable to electromagnetic waves so that a window-type opening must be provided for communication between intracorporeal and extracorporeal electronic components.

For example, from the state of the art in document DE 698 22 310 T2, an optical window arrangement is known for an implantable medical device comprising a housing with a window-type opening provided thereon, a clamp mounted in the window-type opening and forming a lens opening, and a lens mounted in the lens opening. Thereby, the lens is fixed by a solder material inside the lens opening of the clamp. However, the biocompatibility of the entire system is limited by the use of a separate solder material. In particular, contact of the solder material with an area outside the respective implantable device cannot be avoided due to the manufacturing process. In addition, the coating of a lens is associated with a relatively high manufacturing effort.

Against this background, the object of the present invention is to provide a housing component, in particular for implantable devices, which ensures an increased degree of biocompatibility with simultaneously sufficient stability and operational safety. In addition, a housing or implantable device with such a housing component as well as a method for its manufacture is to be provided.

With regard to the housing component, the aforementioned problem has been solved by the subject matter of claim 1. A housing having such a housing component is the subject matter of claim 5 and an implantable device having such a housing component is the subject matter of claim 7. A method of manufacturing a housing component is the subject matter of claim 12.

A housing component according to the invention comprises at least one housing wall section which is equipped with a window-type opening for conducting electromagnetic waves therethrough. Such a housing wall section is preferably a component that can withstand high mechanical loads. However, the material of such structural components is usually impermeable to electromagnetic waves. For this reason, a separate window-type opening is provided to allow the local conduction of electromagnetic waves therethrough. During operation of the housing component according to the invention, for example when used for implantable devices, this ensures high mechanical stability on the one hand and the transmission of data by electromagnetic waves on the other hand.

According to the invention, it is now provided that the window-type opening is hermetically sealed by shaping a blank of a material permeable to electromagnetic waves. The permeability of the blank material to electromagnetic waves allows the conduction of electromagnetic waves therethrough for wireless data transmission even after the window-type opening has been closed. In addition, the hermetic seal ensures the necessary safety during operation of the housing component. If, for example, the housing component is used to encapsulate electronic components in implantable devices and the said electronic components contain materials with high toxicity, such a hermetic seal is necessary to prevent poisoning of the respective organism. On the one hand, the production of the hermetic seal can thus be achieved with only little effort by shaping a blank. On the other hand, hermetic sealing by shaping a blank can reduce the number of materials used, which has a positive effect on the biocompatibility of the housing component. For example, a blank material with high biocompatibility can be used which creates a hermetic seal of the window-type opening by shaping without the use of further materials. In this way, a housing component can be produced, particularly for use in implantable devices which ensures a high degree of biocompatibility with sufficient stability and operational reliability.

In a particularly preferred manner, the shaped blank is configured to enable data transmission by electromagnetic waves in the visual wavelength range from about 400 nm to 700 nm and/or in the near and medium infrared wavelength range from about 700 nm to 50 μm. In particular, the shaped blank is configured to enable data transmission by electromagnetic waves in the range from 800 nm to 1000 nm. In addition, the shaped blank can be configured for the transmission of radio waves, for example, Bluetooth technology at a frequency of approx. 2 GHz, for data transmission between implants and extracorporeal units. If, however, a metal material is used for the housing wall section, radio waves can cause undesired heating through induction and eddy currents. Radio data transmission could be attenuated.

According to a preferred embodiment of the housing component, the shaped blank is made of a non-metallic and/or amorphous and/or inorganic material, in particular with silicon as the main component. The shaped blank is preferably made of a glass material. Such materials can exhibit a high degree of permeability to electromagnetic waves and at the same time a high degree of biocompatibility. In addition, glass materials for the seal of a suitably dimensioned window-type opening, for example, have a high degree of break resistance when the glass thickness is sufficiently dimensioned. Alternatively, it is also possible to use blanks made of polymer materials which are disadvantageous to glass materials in terms of the hermetic encapsulation to be achieved, however, can be easily shaped.

Further preferably the housing wall section consists of a metal material, in particular titanium or a titanium alloy. Metal materials have a high degree of strength and, thus, a high degree of break resistance. By using metal materials, the safety of the encapsulation of an implantable device can be guaranteed. Titanium is also highly biocompatible and is in particular not recognized as a foreign body by human bodies.

According to a preferred embodiment of the housing component, the hermetic seal is produced by hot forming of the blank, for example by forming at a temperature in the range of 600° C. to 800° C. When using blanks made of a glass material, a sufficiently reduced viscosity can be ensured by forming at such temperatures.

According to a particularly preferred embodiment, a material-locking connection is established between the shaped blank and the window-type opening, in particular by chemical reaction of the material pair. A hermetic sealing of the window-type opening can be realized in a particularly advantageous way by such a material-locking connection. The material-locking connection can, for example, be produced by reacting a titanium oxide layer forming the surface of the housing wall section with the glass material of the shaped blank.

It can also be advantageous if the shaped blank is connected to the window-type opening in a force-locking and/or form-fit manner. For example, it is possible to produce a press bond by shrinking the housing wall section onto the shaped blank. The shaped blank can also engage behind the window-type opening on one or both sides with a collar-shaped projection. The connection security between the shaped blank and the housing wall section can thereby be improved.

In a particularly preferred way, the connection between the shaped blank and the window-type opening is made exclusively by shaping the blank, in particular free of further connecting elements or materials. For example, the use of a separate solder or adhesive material or a bonding agent for fixing a lens within a window-type opening of a housing can be completely dispensed with. In particular, this allows the use of materials that restrict biocompatibility to be kept to a minimum or to be completely avoided. The biocompatibility of the overall system can thus be further improved.

According to another preferred embodiment, the shaped blank forms an optical window element which hermetically seals the window-type opening. Such an optical window element can, for example, be adapted to the respective application with regard to its optical properties, for example by influencing the beam path of the electromagnetic waves according to the specific application. For example, the optical window element can be lens-shaped. Such a lens shape can favorably influence the beam path of electromagnetic waves and, thus, improve transmission quality. For this purpose, the upper and/or lower side of the optical window element can form a curved surface. It can also be advantageous, if the optical window element is plate-shaped, especially with flat upper and lower sides. Such a plate-shaped window element can be manufactured with little effort and also allows the production of an almost uninterrupted or flat outer shape of the housing component.

According to a further advantageous embodiment, the housing wall section is configured to be connected to at least one further housing element, in particular for the limitation and/or hermetic encapsulation of a housing interior. For this purpose, the housing wall section may, for example, be provided with suitable fastening sections or fastening elements which enable or favor a material-locking connection with the respective further housing element. Such fastening sections or fastening elements can also be designed in addition to the force-locking and/or form-fit connection with another housing element. This can support the maintenance of a material-locking connection.

In another preferred way, the housing wall section forms a housing upper part, in particular a housing cover or a housing upper shell. In particular, the housing wall section is configured to be connected to a housing lower part, preferably a housing lower shell. The formation of the housing wall section as a housing cover or housing upper shell increases the design scope for the housing lower shell, since the latter no longer necessarily has to be provided with a window-type opening.

It can furthermore be advantageous, if the window-type opening of the housing wall section is accessible for applying force to both sides of the blank. This further reduces the manufacturing effort. In this case, in particular, relatively easily constructed equipment can be used to form the blank so that only low manufacturing costs are incurred. Accessibility of the window-type opening from both sides can be achieved, for example, by a plate-shaped, in particular flat, configuration of the housing wall section. Such accessibility is also ensured when the housing wall section is formed with only a small curvature or a small “three-dimensional shape”.

According to a further aspect of the invention, a housing is provided, in particular for implantable devices, with at least one housing component described above, which is connected to at least one further housing element. In a housing according to the invention with a housing component described above, a high degree of biocompatibility is ensured with simultaneous sufficient stability and operational reliability. The window-type opening of the housing component, closed by the shaped blank, guarantees wireless data transmission with simultaneous hermetic sealing. In addition, the hermetic seal produced by shaping a blank can guarantee improved biocompatibility due to the limitation of the materials required.

In a preferred manner, the housing component and the further housing element limit the housing interior. Such a housing interior can be configured and/or dimensioned in particular to accommodate the components required in each case. This includes, for example, active electronic components. Suitable fastening elements can be provided on the housing component and/or the additional housing element to accommodate and/or fix such components inside the housing. As described above, the housing component of the housing can preferably be configured as the upper part of the housing and the further housing element as the lower part of the housing.

In order to avoid poisoning by toxic materials in the electronic components used in each case, the housing interior, which is limited by the housing component and at least one further housing element, is hermetically encapsulated in a particularly preferred manner. For this purpose, the housing components and the further housing element can be welded together, preferably without the aid of further materials or also biocompatible materials.

According to a further aspect of the present invention, an implantable device is provided, in particular for medical applications, with at least one housing component described above or a housing described above. As already described with regard to the housing component described above and the housing described above, a high degree of biocompatibility is ensured even with an implantable device configured in accordance with the invention, while at the same time ensuring sufficient stability and operational reliability. Due to the wireless data transmission, the window-type opening of the housing component, which is closed with the shaped blank, is guaranteed, whereby the hermetic seal of the window-type opening at the same time ensures the prevention of poisoning of the respective organism by the toxicity of possible electronic components in the housing interior. Finally, the biocompatibility of the implantable device is improved by the fact that the hermetic seal produced by shaping a blank can be achieved with a reduced number of materials required.

In another preferred way, a communication module is provided, which is further preferred for transmitting and/or receiving electromagnetic waves through the window-type opening closed with the shaped blank. This allows wireless data transmission and, thus, relatively easy use of the implantable device in living organisms.

In a particularly preferred way, the communication module is configured to enable data transmission by electromagnetic waves in the visual wavelength range from about 400 nm to 700 nm and/or by electromagnetic waves in the near and medium infrared wavelength range from about 700 nm to 50 μm. In particular, the communication module is configured as an infrared module which is designed for data transmission in the infrared range at wavelengths from 800 nm to 1000 nm. Data transmission in the infrared range at wavelengths from 800 nm to 1000 nm enables a relatively high transmission rate due to the high frequencies. In addition, organic tissue is sufficiently permeable in this wavelength range so that an infrared module of this type allows interference-free transmission between intra- and extracorporeal components. Apart from this, it is also possible to provide data transmission in other wavelength ranges, for example, by radio waves in the kilohertz or megahertz range, and to configure the communication module accordingly. For example, Bluetooth technology at a frequency of about 2 GHz could be considered here. As mentioned above, however, when using a metal material for the housing wall section or for the entire housing, the use of radio waves due to induction and eddy currents can generate undesired heating. Radio data transmission can be attenuated.

According to a further embodiment of the implantable device, an electronic module is provided, preferably for controlling the communication module. Communication module and electronic module can be configured as a unit or as separate components.

It can further be advantageous, if the communication module and/or the electronic module is fixed inside the housing, especially at the housing component or at the further housing element. This ensures that the module is securely positioned so that the risk of damage to the respective module is reduced. As already described above, suitable fastening elements can be provided on the housing component and/or the further housing element for fixing electronic components in the housing interior. Finally, the electronic module can, in a further preferred manner, enclose the communication module at least in sections, whereby the electronic module preferably has a recess through which the communication module is conducted. This enables a space-saving arrangement in the housing interior to be achieved with little effort.

Finally, the present invention relates to a method for manufacturing a housing component, in particular for implantable devices. According to the method according to the invention, initially a housing wall section with a window-type opening is provided for the transmission of electromagnetic waves and then the window-type opening is hermetically sealed by shaping a blank made of a material permeable to electromagnetic waves. Such a method allows the production of a housing component described above.

In a preferred embodiment of the method, the blank is positioned within the window-type opening of the housing wall section. The arrangement of housing wall section and blank is then preferably positioned on a die. In addition, the housing wall section can first be arranged on the die and then the blank can be positioned in the window-type opening.

The arrangement is further preferably heated, especially to a temperature between 600° C. and 800° C., especially preferred to 650° C. After reaching the desired temperature, the blank is shaped by a punch into an optical window element. The window-type opening is hermetically sealed by the shaping process, in particular by forming a material-locking connection between the housing wall section and the shaped blank. The punch can then be removed from the shaped blank again. The thus produced housing component can then be cooled and subsequently removed from the die. The shape of the die and punch can be selected according to the desired final geometry of the shaped blank. If, for example, flat upper and lower sides of the shaped blank are desired, the die and punch must also be selected accordingly. On the other hand, a lens shape of the formed blank requires curved effective surfaces of the die and/or punch.

For a housing component according to the invention or for a method for manufacturing same, for example, a blank made of a glass material with the following composition is used:

SiO₂: 70-80% by weight

Na₂O+K₂O: 10-20% by weight

CaO+MgO: 7-13% by weight

and a remainder of unavoidable impurities.

Such a composition of the glass material can form a particularly advantageous bond with a titanium oxide layer which can be formed on the surface of the housing wall section.

During the shaping process, a pressure of 5 to 15 MPa, particularly 10 MPa, can be advantageously generated at the contact surface between the blank to be shaped and the housing wall section during the shaping process to form a material-locking connection.

Further preferably, the coefficients of thermal expansion for the blank and the housing wall section can be identical or essentially identical. In this way, undesired stresses after cooling can be avoided. For example, the coefficients of thermal expansion of the blank and the housing wall section can be 9.0×10⁻⁶K⁻¹. The coefficients of thermal expansion may also differ from each other in order to produce a press bond after cooling, if this is desired in the respective case.

Further embodiments of the present invention arise from the combination of the features disclosed in the claims, the description and the figures. The present invention is explained in more detail below by means of embodiments and associated drawings.

Therein:

FIG. 1 shows a schematic perspective view of a housing for implantable devices according to an embodiment of the invention.

FIG. 2 shows a schematic cross-sectional view of a housing for implantable devices according to a further embodiment of the invention.

FIG. 3 shows a schematic view of the method step for manufacturing a housing component according to an embodiment of the invention.

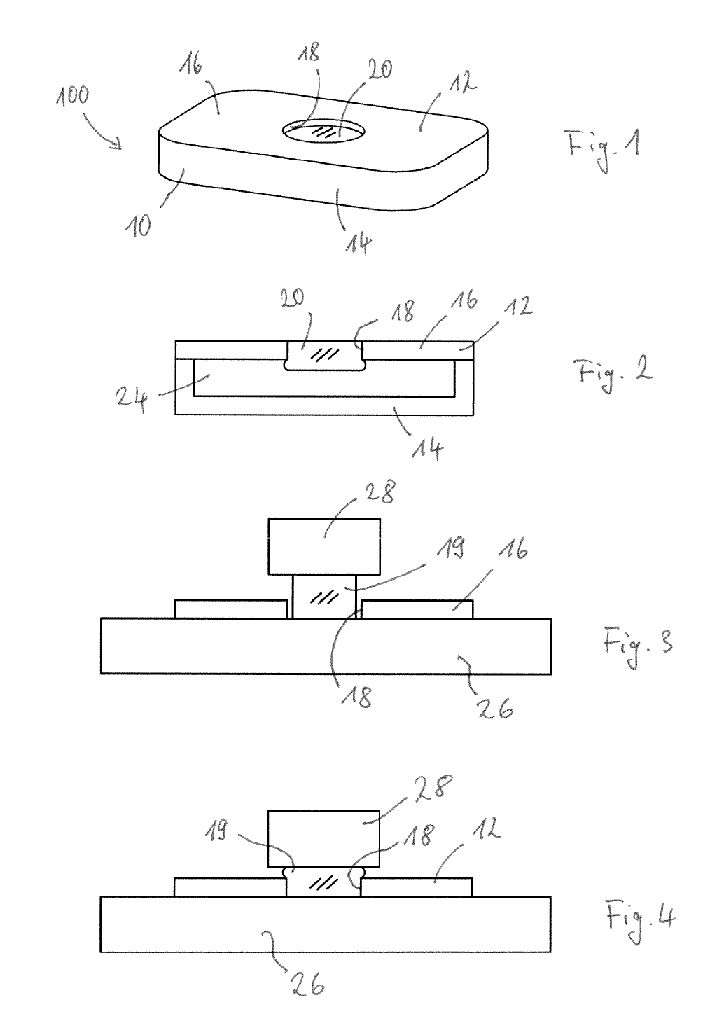

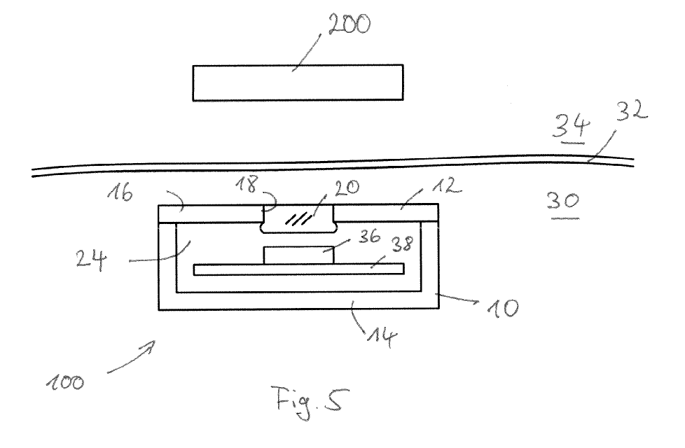

FIG. 4 shows a schematic view of a further method step for manufacturing a housing component according to an embodiment of the invention.

FIG. 5 shows a schematic cross-sectional view of an implantable device in implanted state according to an embodiment of the invention and an extracorporeal unit.

FIG. 1 shows a schematic perspective view of a housing 10 for an implantable device 100 according to an embodiment of the invention. The housing 10 has a housing component 12 which is exemplarily configured here as a housing upper part or housing cover. In addition, the housing 10 has a housing lower part 14 which is exemplarily configured as an example of a housing lower shell. The housing component 12 consists of a housing wall section 16 which is equipped with a window-type opening 18 for conducting electromagnetic waves through. The window-type opening 18 is hermetically sealed by shaping a blank of a material permeable to electromagnetic waves.

The shaped blank preferably consists of a glass material, in particular with the composition below:

SiO₂: 70-80% by weight

Na₂O+K₂O: 10-20% by weight

CaO+MgO: 7-13% by weight

and a remainder of unavoidable impurities.

In a particularly preferred manner, the glass material has the following composition:

SiO₂: 75% by weight

Na₂O+K₂O: 15% by weight

CaO+MgO: 10% by weight

and a remainder of unavoidable impurities.

The housing wall section 16 preferably consists of a metal material, in particular titanium or a titanium alloy. The surface of the housing wall section 16 is formed in particular by a titanium oxide layer.

The hermetic seal between the shaped blank 20 and the housing wall section 16 is preferably produced by a material-locking connection, preferably by a chemical reaction between the SiO₂ of the glass material and the titanium oxide layer of the housing wall section 16.

As can be derived from the cross-sectional view in FIG. 2, the shaped blank 20 can also be connected in a form-fit manner to the housing wall section 16. For this purpose, a collar-shaped projection 22 can be formed by shaping, which engages behind the window-type opening 18. In addition, collar-shaped projections can also be formed on both sides of the shaped blank 20 to engage behind the window-type opening on both sides, which is not shown here. It can also be advantageous, if the shaped blank 20 is connected in a force-locking manner to the window-type opening 18, for example, by shrinking the housing wall section 16 onto the shaped blank 20. The coefficients of thermal expansion can be appropriately selected for this. If stresses are to be avoided in the shaped blank 20, the coefficients of thermal expansion of the two materials can be identical or essentially identical.

As can be further derived from FIG. 2, the connection between the shaped blank 20 and the window-type opening 18 of the housing wall section 16 is made exclusively by shaping the blank, in particular free of other connecting elements or materials. The shaped blank 20 preferably forms an optical window element which hermetically seals the window-type opening 18. The optical window element formed by the shaped blank 20 is plate-shaped, in particular with flat upper and lower sides. In addition, the optical window element formed by the shaped blank 20 can be lens-shaped, in particular with at least one curved surface, which is not shown in the Figures.

It can further be derived from FIG. 2 that the housing wall section 16 is connected to the other housing element 14 and limits a housing interior 24. The housing interior 24 is hermetically encapsulated, in particular by material-locking connection of the housing wall section 16 and the further housing element 14. For this purpose, the housing wall section 16 may, for example, be provided with fastening sections or fastening elements not shown here which enable or favor material-locking connection with the respective further housing element 14. Such fastening sections or fastening elements can also be configured in addition to the force-locking and/or form-fit connection with another housing element, whereby the maintenance of a material-locking connection can be supported.

From FIGS. 3 and 4, it can be seen a schematic method sequence for the manufacture of a housing component 12 according to the invention. In a first step, a housing wall section 16 with a window-type opening 18 is provided for conducting electromagnetic waves therethrough. A blank 19 of a material permeable to electromagnetic waves is positioned within the window-type opening 18 of the housing wall section 16. The arrangement of housing wall section 16 and blank 19 is then preferably positioned on a die 26. In addition, the housing wall section 16 can first be arranged on the die 26 and then the blank 19 can be positioned in the window-type opening 18.

In a further step, the arrangement of housing wall section 16 and blank 19 is further heated, in particular to a temperature between 600° C. and 800° C., particularly preferred to 650° C.

After the desired temperature has been reached, the window-type opening 18 is hermetically sealed, as shown in FIG. 4, by shaping the blank 19, in particular by forming a material-locking connection between the housing wall section 16 and the shaped blank 20. For this purpose, the blank 19 is shaped into an optical window element by a punch 28. After the shaping process, the punch 28 can be removed again from the shaped blank 20. The thus produced housing component 14 can then be cooled and subsequently removed from the die 26.

FIG. 5 shows a schematic cross-sectional view of an implantable device 100 with a housing 10 according to an embodiment of the invention. The implantable device 100 is shown in an implanted state, thus, in an area 30 below a skin section 32. FIG. 5 also shows an extracorporeal unit 200 which is arranged in particular in an area 34 outside the body. The extracorporeal unit 200 can in particular be an extracorporeal communication unit.

Furthermore, a communication module 36, which is preferably designed as an infrared module, is arranged inside the housing interior 24. Data transmission in the infrared range at wavelengths from 800 nm to 1000 nm enables a high transmission rate with low susceptibility to interference. The communication module 36 can send and receive data through the closed window-type opening 18. Furthermore, an electronic module 38 is provided within the housing interior 24 of the implantable device 100, preferably for controlling the communication module 36. Communication module 36 and electronic module 38 can be configured as a unit or as separate components. The electronic module 38 can, for example, be configured as a circuit board.

A housing component according to the invention or a housing equipped with the same may be used in particular for medical applications, for example, for implantable devices used for therapeutic and/or diagnostic purposes. As an example only, pacemakers can be considered as a possible application for a housing component according to the invention or a housing equipped with the same. However, the invention is not limited to medical applications. A housing component or a housing equipped with the same can also be used in vacuum technology.

1. A housing component for implantable devices, the housing component comprising:
at least one housing wall section that includes a window-type opening for conducting electromagnetic waves therethrough, wherein said window-type opening is hermetically sealed as a result of a blank being shaped that is made of a material permeable to electromagnetic waves.

2. The housing component according to claim 1, wherein the shaped blank is made of a non-metallic and/or amorphous and/or inorganic material, with silicon as a main component, the shaped blank further being made of a glass material and/or wherein the housing wall section is made of a metal material including titanium or a titanium alloy.

3. The housing component according to claim 1, wherein the hermetic seal is produced by hot-forming the blank and/or by a material-locking connection between the shaped blank and the window-type opening, by chemical reaction of the material pair, and/or wherein the shaped blank is connected in a force-locking and/or form-fitting manner to the window-type opening and/or wherein the connection between the shaped blank and the window-type opening is produced exclusively by shaping the blank, free from further connecting elements or materials, and/or wherein the formed blank forms an optical window element which hermetically seals the window-type opening, and/or wherein the optical window element is lens-shaped, with at least one curved surface, or plate-shaped, with flat upper and lower sides.

4. The housing component according to claim 1, wherein the housing wall section is configured to be connected to at least one further housing element, for limiting and/or hermetically encapsulating a housing interior, and/or wherein the housing wall section forms a housing upper part, including a housing cover or a housing upper shell, and/or wherein the housing wall section is configured to be connected to a housing lower part, including a housing lower shell, and/or wherein the window-type opening of the housing wall section is accessible for applying force to the blank on both sides.

5. The housing component according to claim 1, wherein the housing component is included in a housing and connected to at least one further housing element.

6. The housing according to claim 5, wherein the housing component and the at least one further housing element delimit and/or hermetically encapsulate a housing interior and/or wherein the housing component is configured as an upper housing shell and the at least one further housing element is configured as a lower housing shell.

7. An implantable device for medical applications, the implantable device comprising:
at least one housing component comprising at least one housing wall section that includes a window-type opening for conducting electromagnetic waves therethrough, wherein said window-type opening is hermetically sealed as a result of a blank being shaped that is made of a material permeable to electromagnetic waves, or a housing comprising the at least one housing component that is connected to at least one further housing element.

8. The implantable device according to claim 7, wherein a communication module is provided which is arranged for sending and/or receiving electromagnetic waves through the window-type opening closed by the shaped blank, and/or wherein the communication module is configured as an infrared module and/or wherein the communication module is fixed inside the housing, the communication module being fixed on the housing component or on the at least one further housing element.

9. The implantable device according to claim 7, wherein an electronic module is provided for controlling the communication module, wherein the electronic module is further fixed inside the housing, on an inner side of the housing component or the housing element, and/or wherein the electronic module encloses the communication module at least in sections, wherein the electronic module has a recess through which the communication module is conducted.

10. A method for manufacturing a housing component for implantable devices, the method comprising:
providing a housing wall section that includes a window-type opening for conducting electromagnetic waves therethrough; and
hermetically sealing the window-type opening as a result of a blank being shaped, the blank being made of a material permeable to electromagnetic waves.

11. The method of claim 10, wherein hermetically sealing the window-type opening includes positioning the blank within the window-type opening and then positioning the housing wall section and the blank on a die.

12. The method of claim 10, wherein hermetically sealing the window-type opening includes arranging the housing wall section on a die and then positioning the blank in the window-type opening.

13. The method of claim 10, further comprising heating an arrangement of the housing wall section and the blank positioned in the window-type opening to a temperature between 600° C. and 800° C.

14. The method of claim 10, wherein shaping the blank includes shaping the blank into an optical window element by a punch.

15. The method of claim 14, further comprising removing the punch from the shaped blank, and cooling the housing component.

16. The method of claim 10, further comprising connecting the housing wall section to a further housing element by a material-locking connection.

17. The implantable device according to claim 7, wherein the blank is made of a non-metallic and/or amorphous and/or inorganic material, and/or wherein the housing wall section is made of a metal material.

18. The implantable device according to claim 7, wherein the blank is made of a glass material and/or silicon.

19. The implantable device according to claim 7, wherein the blank forms an optical window element that hermetically seals the window-type opening, wherein the optical window element is lens-shaped with at least one curved surface.

20. The implantable device according to claim 7, wherein the blank forms an optical window element that hermetically seals the window-type opening, wherein the optical window element is plate-shaped with flat upper and lower sides.
